(19) United States
(12) Patent Application Publication
(10) Pub. No.: US 2025/0012779 A1
(43) Pub. Date: Jan. 9, 2025

(54) QUANTITATIVE METHODS USING VOLATILE ORGANIC COMPOUNDS IN EXHALED BREATH FOR THE IDENTIFICATION OF INFLUENZA INFECTIONS

(71) Applicant: Ventura Prototypes, LLC, Albuquerque, NM (US)

(72) Inventor: Brent Holbrook, Albuquerque, NM (US)

(21) Appl. No.: 18/747,913

(22) Filed: Jun. 19, 2024

Related U.S. Application Data
(60) Provisional application No. 63/511,802, filed on Jul. 3, 2023.

(51) Int. Cl. G01N 33/497; G01N 27/12

(52) U.S. Cl. CPC G01N 33/4975; G01N 27/128

(57) ABSTRACT

A method for preparing, analyzing and characterizing volatile organic compounds (VOCs) from exhaled breath of humans that is useful for identifying a presence of an influenza infection, can include extracting exhaled breath samples from subjects to obtain a presence of specific VOCs or VOC(s) concentration levels, and producing isolated or measurable VOC concentrations. The measuring process can include VOC reaction reagents to identify or measure VOC concentration level, selectively measuring attributes associated with VOCs, measuring voltage differences using voltage measuring sensors or resistance sensor, or selectively using size-based filtration to identify VOCs of interest, and analyzing exhaled breath samples produced.

This application claims priority of provisional application No. 63/511,802, filed Jul. 3, 2023, the entire contents of which is incorporated by reference.

### TECHNICAL FIELD

Collection, analysis and identification of volatile organic compounds (VOCs) sampled from exhaled breath in humans are useful for the characterization and identification of influenza viral infections.

### BACKGROUND

Influenza virus in humans is one of the most common infectious diseases worldwide. Current standards of diagnostics methods are invasive and less accurate when used in a point of care setting. Diagnostic identification typically requires culturing the virus from the upper respiratory tract.

Seasonal influenza has historically caused significant morbidity and mortality, year over year, not only in the United States but throughout the globe. Point-of-Care (POC) testing using rapid influenza diagnostic tests (RIDTs), immunoassays that detect viral antigens, are often used for diagnosis by physician offices, urgent care centers, and emergency rooms. These tests are rapid but significantly lack sensitivity, which is estimated to be as low as 50%. RIDTs, also known as flu antigen tests, are the most common type of influenza test. They are immunoassays that detect the presence of influenza A and B viral antigens in respiratory specimens, and can provide results in about 10-15 minutes. RIDTs work by detecting antigens, or parts of the virus, that trigger an immune response. The test usually involves inserting a swab into the nostril to obtain a sample. RIDTs are approved for specific types of respiratory specimens, but are not recommended for use in hospitalized patients with suspected influenza. Instead, molecular assays, such as RT-PCR, are recommended for hospitalized patients because they are more sensitive and specific.

RIDTs have been widely used since the 1990s because they are easy to use, provide quick results, and are suitable for point of care testing. However, issues with their diagnostic sensitivity have been known for decades, and their poor performance during the 2009 influenza A (H1N1) pandemic drew increased attention to these issues. That is, these tests are rapid but significantly lack sensitivity, which is estimated to be as low as 50%.

Alternatively, laboratory testing by polymerase chain reaction (PCR) is highly sensitive and specific. PCR is a molecular assay that can detect influenza viral RNA or nucleic acids in respiratory specimens. PCR is the preferred method for detecting influenza in clinical specimens because it is faster, more sensitive, specific, and cost-effective than other methods, including culture. PCR can also detect influenza before diagnosis by immunological methods. Still, historically these assays have been performed in centralized clinical laboratories necessitating specimen transport, increasing the time to result, and significantly increasing the overall cost of delivering healthcare for those with influenza-like symptoms.

Volatile organic compounds (VOCs) are produced in the human body via various metabolic pathways, and the presence of influenza viruses affect these metabolic pathways. However, these production pathways of VOCs have shown to be used as a potential diagnostic biomarker for many other infectious diseases. The presence of abnormal disease-associated metabolites within clinical samples can provide information about the development of a disease, and the chemical signatures of VOCs in humans can be utilized for diagnosis. For this reason, there is a high interest in developing non-invasive analytical processes and methods to analyze the VOCs profile in exhaled breath.

In recent years, research around breath analysis for identifying, diagnosing, and routine monitoring metabolic disorders or by a specific infectious disease has become more popular due to its inherent noninvasiveness. The use of exhaled breath as a biomarker for disease identification has been explored since the times of Hippocrates and ancient Greece.

Nonetheless, this technique has not been clinically helpful for several reasons. Most specifically, the lack of sensitive enough technology to identify specific VOCs, the systematic understanding of VOC production during a viral infection and the development of standard processes used to collect and analyze exhaled breath samples.

There is accordingly a need for tests that can rapidly and inexpensively detect influenza not only in clinical settings, but also out in the field.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide a method for preparing, analyzing and characterizing volatile organic compounds (VOCs) from exhaled breath of subjects that is useful for identifying a presence of an influenza infection, that includes (a) extracting exhaled breath samples from subjects to obtain a presence of specific VOCs or VOC(s) concentration levels; and (b) producing isolated or measurable VOC concentrations extracted by: (i) VOC reaction reagents to identify or measure VOC concentration level, (ii) selectively measuring attributes associated with VOCs, (a) measuring voltage differences using voltage measuring sensors or resistance sensor, or (b) selectively using size-based filtration to identify VOCs of interest, and (c) analyzing exhaled breath samples produced.

In the disclosure, a positive result for influenza is a ratio of about 2.29 sample measurement/ambient air measurement, and an accuracy in a range of about 79% to about 85% for influenza. The VOCs may be at least one selected from acetaldehyde, propanal, n-propyl acetate, methyl-methacrylate, styrene, 1,1-dipropoxypropane, isoprene, acetone, ethyl acetate, butanone, 1-butene, dimethyl sulfide, ethylene or hexanal.

In the disclosure, the VOCs are detected by resistance measurement, or VOCs are detected by resistance measurement, and the resistance measurement shows peaks at about 131 seconds, about 209 seconds and about 290 seconds. The breath samples may be collected by exhaling into a polyvinyl fluoride gas sampling bag, and the breath samples are selectively bound to carbon nanotubes.

The disclosure may further include selectively binding the breath samples to carbon nanotubes; measuring a baseline resistance; and comparing the baseline resistance to resistance changes after sample collection. The voltage differences may be measured using metal-oxide semiconductors, conductive polymers or polymer composites containing carbon black.

The disclosure, in part, pertains to a method for preparing, analyzing and characterizing volatile organic compounds (VOCs) from exhaled breath of humans for identifying a presence of an influenza infection, that includes: (a) extracting exhaled breath samples from subjects to obtain a presence of total VOC concentration levels; and (b) Selectively measuring attributes associated with the total VOCs by measuring voltage differences using voltage measuring sensors or resistance sensors.

In the disclosure, a positive result for influenza is a ratio of about 2.29 sample measurement/ambient air measurement, and an accuracy in a range of about 79% to about 85% for influenza. The VOCs may be at least one selected from acetaldehyde, propanal, n-propyl acetate, methyl-methacrylate, styrene, 1,1-dipropoxypropane, isoprene, acetone, ethyl acetate, butanone, 1-butene, dimethyl sulfide, ethylene or hexanal.

In the disclosure, the VOCs are detected by resistance measurement, or VOCs are detected by resistance measurement, and the resistance measurement shows peaks at about 131 seconds, about 209 seconds and about 290 seconds. The breath samples may be collected by exhaling into a polyvinyl fluoride gas sampling bag, and the breath samples are selectively bound to carbon nanotubes.

The disclosure may further include selectively binding the breath samples to carbon nanotubes; measuring a baseline resistance; and comparing the baseline resistance to resistance changes after sample collection. The voltage differences may be measured using metal-oxide semiconductors, conductive polymers or polymer composites containing carbon black.

### BRIEF DESCRIPTION OF THE DRAWINGS

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1 is a graph showing the measurement of the resistance (μΩ) of acetaldehyde as a function of time (seconds) in accordance with an aspect of the disclosure.

FIG. 2 is a graph showing the measurement of the resistance (μΩ) of propanol as a function of time (seconds) in accordance with an aspect of the disclosure.

FIG. 3 is a graph showing the measurement of the resistance (μΩ) of n-propyl acetate as a function of time (seconds) in accordance with an aspect of the disclosure.

FIG. 4 is a graph showing the measurement of the resistance (μΩ) of methyl methacrylate as a function of time (seconds) in accordance with an aspect of the disclosure.

FIG. 5 is a graph showing the measurement of the resistance (μΩ) of styrene as a function of time (seconds) in accordance with an aspect of the disclosure.

FIG. 6 is a graph showing the measurement of the resistance (μΩ) of 1,1-dipropoxypropane as a function of time (seconds) in accordance with an aspect of the disclosure.

FIG. 7 is a graph showing the measurement of the resistance (μΩ) of isoprene as a function of time (seconds) in accordance with an aspect of the disclosure.

FIG. 8 is a graph showing the measurement of the resistance (μΩ) of acetone as a function of time (seconds) in accordance with an aspect of the disclosure.

FIG. 9 is a graph showing the measurement of the resistance (μΩ) of ethyl acetate as a function of time (seconds) in accordance with an aspect of the disclosure.

FIG. 10 is a graph showing the measurement of the resistance (μΩ) of butanone as a function of time (seconds) in accordance with an aspect of the disclosure.

FIG. 11 is a graph showing the measurement of the resistance (μΩ) of 1-butene as a function of time (seconds) in accordance with an aspect of the disclosure.

FIG. 12 is a graph showing the measurement of the resistance (μΩ) of dimethyl sulfoxide as a function of time (seconds) in accordance with an aspect of the disclosure.

FIG. 13 is a graph showing the measurement of the resistance (μΩ) of ethylene as a function of time (seconds) in accordance with an aspect of the disclosure.

FIG. 14 is a graph showing the measurement of the resistance (μΩ) of hexanal as a function of time (seconds) in accordance with an aspect of the disclosure.

### DETAILED DESCRIPTION

The particular values and configurations discussed in the following non-limiting examples can be varied and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

The technology of the present disclosure utilizing exhaled breath as a biomarker for influenza infections. Historically, exhaled breath has not been practical in a clinical setting because of the need for specialized equipment and training by clinical staff. Additionally, the hardware needed to facilitate VOC testing has not been practical to use as a point-of-care test. However, the proposed “e-nose” technology is believed to measure VOCs in the needed parts per billion range and can be utilized through an application on a smartphone or tablet.

In recent years, research around breath analysis for identifying, diagnosing, and routine monitoring of metabolic disorders or a specific disease has become more popular due to its inherent noninvasiveness. However, the potential relationship between VOCs emitted by the human body and influenza viral infections has not been significantly established. The use of exhaled breath as a potential biomarker for disease identification has been explored since the times of Hippocrates and ancient Greece. Nonetheless, this technique has not been clinically helpful for several reasons. Most specifically, the lack of sensitive enough technology and the systematic understanding of VOC production during a disease state.

Volatile organic Compounds (VOCs) are produced in the human body via various metabolic pathways, and the presence of diseases affects these metabolic pathways. However, these production pathways of VOCs and metabolic processes are not well established, and there has not been significant research to fully understand metabolic pathways producing VOCs in humans during a viral infection. The presence of abnormal disease-associated metabolites within clinical samples, in principle, can provide information about the development of a disease, and the chemical signatures of VOCs in humans can be utilized for diagnosis. For this reason, there is a high interest in developing non-invasive analytical methods to analyze the VOCs profile in exhaled breath.

Various sensing techniques can detect volatile organic compounds in exhaled breath. Because of the high variability of measurement tools used across the industry, it becomes difficult to establish firm correlations between influenza infections and VOC profile signatures. Furthermore, these compounds may be produced by cellular metabolic processes or inhaled/absorbed from exogenous sources. Influenza viruses and the cells infected with such viruses may produce and/or process these compounds differently than normally functioning human cells. The differences in how these compounds are metabolized can be detectable in exhaled breath. Additionally, analysis using ambient air is a novel approach to understanding how the body metabolizes VOCs while controlling for exogenous sources from the external atmosphere.

The technology of the disclosure identified a gap between traditional RIDTs and PCR assays using volatile organic compounds (VOCs) in exhaled breath as a biomarker for influenza infections. Historically, exhaled breath has not been practical in a clinical setting because of the need for specialized equipment and training by clinical staff. However, utilizing and measuring VOCs in the needed parts per billion range and can be utilized through an application on a smartphone, tablet, or computer has been a process shown to standardize the overall exhaled breath collection process, allowing the use of VOCs to become a legitimate biomarker used for the identification of influenza infections.

Viral and bacterial infections interact with host cells and have a direct effect to the host cell metabolism. Different pathogenic species have been found to produce characteristic profiles of VOCs by virtue of their distinct metabolisms. Specifically, influenza A, influenza B virus and many other subvariants has a direct influence on:
1. glycolysis
2. glucogenesis
3. the Krebs Cycle
4. fatty acid synthesis
5. glycogenesis
6. the Pentose Phosphate Pathway
7. the electron transport chain
8. the Cori cycle.

The process of collecting, analyzing and characterizing collected VOCs from exhaled breath for the purpose of identifying an influenza infection is currently not a standard of practice for point of care testing. This fact has resulted from the many types of breath analyzers developed and many point of care tests are not digital in nature. The quantitative methods described here, have overcome many of the difficulties associated with influenza, breath-based diagnostics by:
1. Directly measuring VOCs in exhaled breath,
2. Directly measuring VOCs in external, ambient air,
3. Using a comparative analysis of directly measured, breath-based VOCs to external, ambient air,
4. Using a comparative analysis of directly measured VOCs to sample temperatures, or
5. Using a comparative analysis of directly measured VOCs to sample humidity.

Volatile Organic Compounds (VOCs) are defined as organic chemical compounds whose composition makes it possible for them to evaporate under normal indoor atmospheric conditions of temperature and pressure.

Ratio analysis measures ratio fluctuations and the magnitude of change, and compares direct measurements of resistance values of any given specified VOC in a way that compares measurements from the ambient air concentrations compared to the measured values of the same VOC when measured from an exhaled breath sample. An example of ratio analysis is for acetaldehyde.

### Example 1

Ambient air measurement of acetaldehyde is at 1.75 parts per billion baseline. Exhaled breath sample measurement of acetaldehyde at 4.0 parts per billion. The calculation was as follows:

$$4. / 1.75 = 2.285 .$$

A ratio of about 2.285 (or 2.29±0.03) would indicate a positive result for a change in acetaldehyde. This process is then repeated for all fourteen VOCs listed. If there are positive results in at least seven of the fourteen VOCs listed, influenza is present in the subject who produced the exhaled breath sample. If six or less positive results of ratio analysis is determined, the presence of influenza is negative for the subject who produced the exhaled breath sample

The diagnostic process of the disclosure was developed using the data generated from a breath-based diagnostic tool that analyses volatile organic compounds (VOC) in exhaled breath. The tool comprises a sampling bag that can be a TEDLAR (polyvinyl fluoride)-gas sampling bag, designed for collection and storage of the exhaled breath from patient samples. The sample bag is then connected directly to the device for sample analysis via plastic tubing. Additionally, the tool used has specific capabilities to measuring the concentration and composition of VOCs in the captured breath sample. The VOC identification process feature of the tool is accomplished by using sensing technology that utilizes carbon-nanotubes that can selectively bind to target VOCs. Once the gas sample has been selectively bound, a baseline electrical resistance is used and compared to measured resistance changes after sample collection. The voltage difference between the known standard resistance levels of the carbon nanotubes and the VOC bound nanotubes can then determine the concentration and specific compound of interest.

### DESCRIPTION OF GRAPHS, FIGURES AND ANALYTIC PROCESS

The data was developed using a NASA (National Aeronautical and Space Administration) developed E-Nose sensing technology. The E-Nose can generate a dynamic bar graph that shows real-time changes, based on normalized values derived from an initial resistance code. Users have the flexibility to reset the graph and modify the initial resistance value whenever necessary, using a “reset graph” option provided in the supporting software. This enables the bar graph to adapt to any subsequent changes detected by the sensor while still recording the sensor data. The bar graph can accommodate up to 64 channels and offers the choice of displaying data on a logarithmic scale, with the ability to adjust scales for all 64 channels through the application. Additionally, the software application includes an email feature that allows users to send sensor data as an attachment via email. The sensor data includes readings, timestamps, and is recorded at regular intervals. The application provides real-time sensor response on a mobile phone or tablet screen. Users can selectively display and leave certain sensor channels as empty channels on the graph. This deselection function is accessible through the iPhone application's graphical user interface (GUI). During operation, the user exhales into the collection device, allowing the device to capture a breath sample. The collected breath sample is then passed through the VOC analysis module, where the concentration and composition of VOCs are measured. The resulting VOC data is fed into the diagnostic algorithm, which interprets the data and generates diagnostic information based on predetermined criteria. The diagnostic information can be displayed to the user or transmitted to a healthcare professional or a remote monitoring system.

The specific setting used to generate the example data utilized application version 1.5.7 of the E-Nose application. The following settings provide information to the sensor and associated valves and pumps to determine the appropriate sequence for how fast to pull the breath sample, when to switch from exhaled breath samples to ambient air samples, the position of valves, and ultraviolet light purification (on or off). Each graph represents the resistance values of the designated sensor channel over the complete sequence of the investigation. Additionally, the graphs were generated under standard temperature and pressure. The humidity of the sample was held constant with a minimum of 50% and a maximum of 80%. Along with maintaining standard temperature and pressure (Temperature: 25 degrees Celsius (298.15 degrees Kelvin) Pressure: 1 atm (101.325 kPa) of both the sample gas and ambient air. The experimental parameters are set forth in Table 1.

TABLE 1
Experimental Conditions

| Sequence | | | | | | |
|---|---|---|---|---|---|---|
| Time | Pump | Valve 1 | Valve 2 | Ultraviolet | Record | Sequence |
| (sec.) | Rate | Position | Position | LED | Data | Name |
| 10 | 96 | 2 | 2 | Off | Of | Initialize |
| | | | | | | System |
| 300 | 240 | 2 | 1 | Off | On | Amb air |
| 60 | 240 | 1 | 2 | Off | On | Breath |
| 300 | 240 | 2 | 1 | Off | On | Amb air |
| 60 | 240 | 1 | 2 | Off | On | Breath |
| 300 | 240 | 2 | 1 | Off | On | Amb air |
| 60 | 240 | 1 | 2 | Off | On | Breath |
| 300 | 240 | 2 | 1 | Off | On | Amb air |
| 10 | 10 | 2 | 2 | Off | Off | System |
| | | | | | | shutdown |

Measurement of the VOCs is accomplished using e-nose (electronic nose) sensors. The enose was developed in order to mimic human olfaction that functions as a non-separative mechanism: i.e. an odor/flavor is perceived as a global fingerprint. Essentially the instrument comprises head space sampling, a chemical sensor array, and pattern recognition modules, to generate signal patterns that are used for characterizing odors. Most electronic noses use chemical sensor arrays that react to volatile compounds on contact: the adsorption of volatile compounds on the sensor surface causes a physical change of the sensor. A specific response is recorded by the electronic interface transforming the signal into a digital value. Recorded data are then computed based on statistical models. Electronic noses include three major parts: a sample delivery system, a detection system, a computing system.

The more commonly used sensors for electronic noses include:
metal-oxide-semiconductor (MOS) devices-metal-oxide-semiconductor sensors contain a metal oxide coating with an electrical resistance that changes in the presence of a target gas. The presence of the target gas can be inferred by measuring the change in the resistance of the metal oxide layer over time.
conducting polymers-organic polymers that conduct electricity.
polymer composites-similar in use to conducting polymers but formulated of non-conducting polymers with the addition of conducting material such as carbon black.
quartz crystal microbalance (QCM)—a way of measuring mass per unit area by measuring the change in frequency of a quartz crystal resonator. This can be stored in a database and used for future reference.
surface acoustic wave (SAW)—a class of microelectromechanical systems (MEMS) which rely on the modulation of surface acoustic waves to sense a physical phenomenon.
Mass spectrometers that can be miniaturized to form general purpose gas analysis device.

### The Process

This process of the disclosure performs the analysis of VOCs in a way that compares compounds present is exhaled breath as well as in ambient air that is helpful as diagnostic tool for the presence of influenza infection in humans.

The VOC analysis module is responsible for measuring the concentration and composition of VOCs in the captured breath sample. It may employ various analytical techniques, such as gas chromatography-mass spectrometry (GC-MS), ion mobility spectrometry (IMS), or sensor arrays, to detect and quantify specific VOCs. The module may consist of sample preparation components, a separation mechanism, a detection system, and data processing capabilities. It is designed to provide accurate and reliable VOC measurements. The collection device is designed to efficiently capture exhaled breath and deliver it to the VOC analysis module. It may include a mouthpiece or a face mask connected to a collection chamber, which ensures the collection of a representative breath sample. The collection device may further incorporate a flow control mechanism to regulate the breath flow rate and a filter to remove particulate matter and contaminants.

Once the VOCs have effectively been collected, quantified and identified from a human sample, as well as external ambient air, a comparison can be performed to determine the magnitude of change between the two samples for each of the VOCs listed below:
1. acetaldehyde
2. propanal
3. n-propyl acetate
4. methyl-methacrylate
5. styrene
6. 1,1-dipropoxypropane
7. isoprene
8. acetone
9. ethyl acetate
10. butanone
11.1-butene
12. dimethyl sulfide
13. ethylene
14. hexanal.

If it is determined that 7 or more of the 14 VOCs listed have a magnitude change between 1.1× and 5× when comparing the exhaled breath sample and the ambient air sample, it can be determined there is a positive result for the presence of influenza virus.

### Process Overview

Early diagnosis and appropriate treatment are crucial in reducing mortality among people suffering from viral infections. Volatile organic compounds are a diverse group of carbon-based chemicals that are present in exhaled breath and many biofluids may be noninvasively collected from human subjects. Different patterns of VOCs have been hypothesized to be correlated with various infectious diseases. Studies have also shown that many infectious diseases, in lab settings, produce or consume specific VOCs that can serve as potential indirect biomarkers that differentiate the presence of a viral infection. However, the standardization of the collection, analysis and characterization of VOCs in exhaled breath has presented many challenges. Specifically, the systematic investigation into classifying VOC signatures for the identification of influenza viral infections.

Additionally, the use of volatile organic compounds (VOCs) as a diagnostic biomarker has been shown as a potential alternative to current diagnostics tools designed to be used at the point-of-care. The present disclosure enables the use of VOCs to be used as a reliable diagnostic biomarker for the identification and diagnosis of influenza A and influenza B viruses, among other influenza subvariants. This is achieved by the use of specific processes and laboratory techniques which utilizes the identification of multiple VOCs present, combination of VOCs present in exhaled breath, and ratio analysis of present VOCs along with the use of utilizing ambient air, sample temperature, and sample humidity. These specific set up techniques have demonstrated a unique approach to indirectly measuring influenza activity and infections.

One of the significant challenges with using chemical signatures associated with exhaled breath is the large variability of measurements and inaccuracy associated with breath biopsy samples. The first objective of the presented process is to provide a qualitatively measurable technique for the analysis and investigation of influenza virus. This standardized process has the ability to significantly decrease variability of VOC measurements and increase the likelihood that a thorough and complete breath biopsy was successfully produced.

The second objective of this process is to ensure consistency of the collection breath samples. Influenza viruses have historically demonstrated significant viral drift year to year. These changes consist of small mutations in the genes of influenza viruses, which subsequently impacts researchers' ability to consistently collect quality breath samples from year to year.

The third objective of this process is to simplify the overall biopsy process. Historically, point of care testing for influenza infections have utilized a nasal swab to collect biopsy samples used in antigen assays. However, the depth of sample collection or approximate depth into the nasal cavity requires in-depth training for the administering personnel and the needed depth to collect nasal samples is highly variable from patient to patient.

The fourth objective of this process is to standardize the repeatability of taking multiple breath biopsy within a limited amount of time and with reusable or disposable equipment. Seasonal influenza presents difficult challenges for medical staff. Historically, emergency rooms, urgent care settings and primary care offices experience an increase in the number of patients

The fifth objective of this process is to standardize the analytical methods used by establishing a comparison using ambient air, sample temperature and sample humidity levels to control for variables that have previously been problematic.

### VOC Measurement Examples

In the drawing figures, the curve resistance (un) has an initial value before introduction of a gas or fluid that will perturb the measured value. A difference, between an initial measured EPV value and a currently measured resistance value will deviate from the baseline with the passage of time as a result of exposure of the sensor to the gas or fluid. This sensor can be a nanosensor using a protocol of measuring the sensor resistance or current after exposure to the gas or fluid, such as is set forth in U.S. Pat. No. 10,566,089.

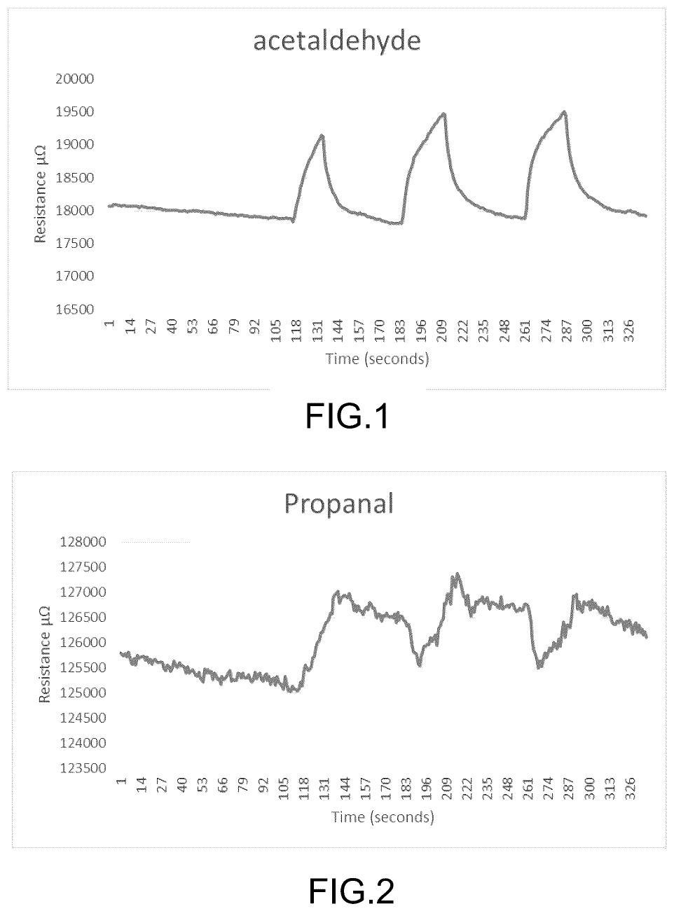

FIG. 1 is a graph showing the measurement of the resistance (μΩ) of acetaldehyde as a function of time (seconds), in accordance with an aspect of the disclosure. As can be seen, three peak maxima can be seen at 131 seconds, 200 seconds and 287 seconds.

FIG. 2 is a graph showing the measurement of the resistance (μΩ) of propanol as a function of time (seconds) in accordance with an aspect of the disclosure. As can be seen, three peak maxima can be seen at 131 seconds, 209 seconds and 290 seconds.

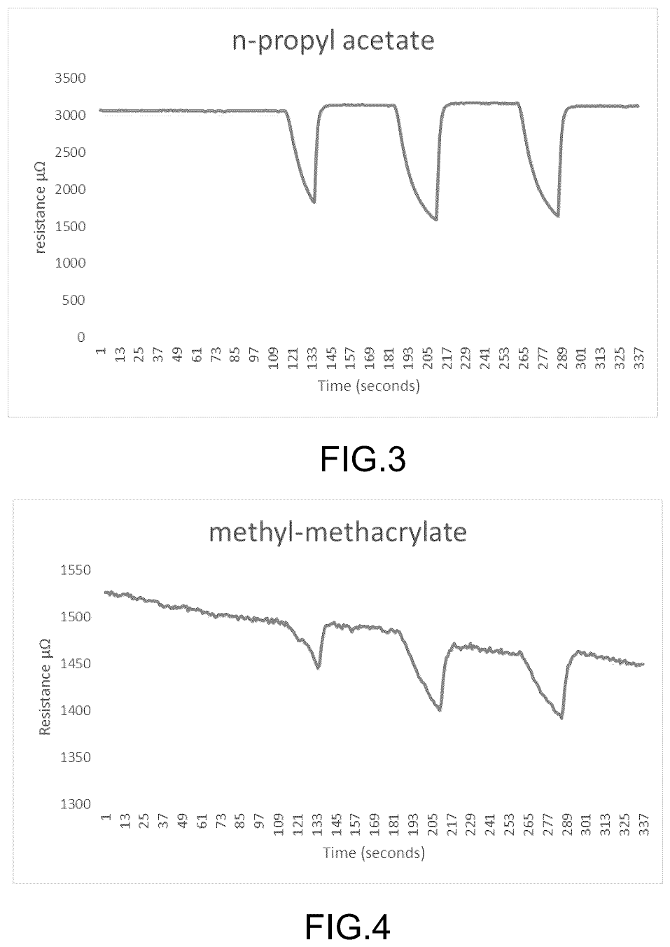

FIG. 3 is a graph showing the measurement of the resistance (μΩ) of n-propyl acetate as a function of time (seconds) in accordance with an aspect of the disclosure. As can be seen, three peak minima can be seen at 131 seconds, 209 seconds and 290 seconds.

FIG. 4 is a graph showing the measurement of the resistance (μΩ) of methyl methacrylate as a function of time (seconds) in accordance with an aspect of the disclosure. As can be seen, three peak minima can be seen at 131 seconds, 209 seconds and 289 seconds.

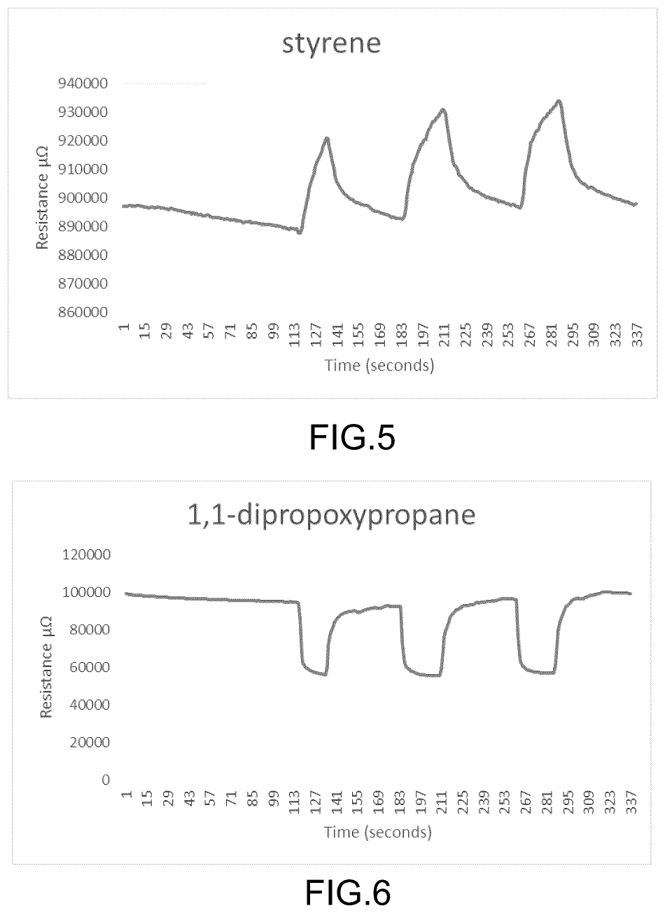

FIG. 5 is a graph showing the measurement of the resistance (μΩ) of styrene as a function of time (seconds) in accordance with an aspect of the disclosure. As can be seen, three peak maxima can be seen at 131 seconds, 209 seconds and 290 seconds.

FIG. 6 is a graph showing the measurement of the resistance (μΩ) of 1,1-dipropoxypropane as a function of time (seconds) in accordance with an aspect of the disclosure. As can be seen, three peak minima can be seen at 131 seconds, 209 seconds and 290 seconds.

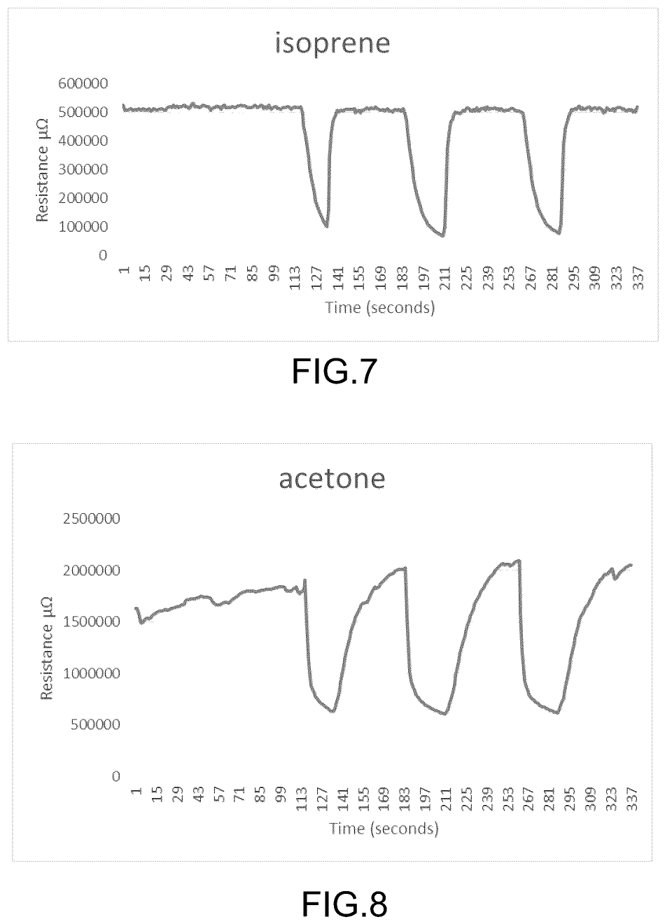

FIG. 7 is a graph showing the measurement of the resistance (μΩ) of isoprene as a function of time (seconds) in accordance with an aspect of the disclosure. As can be seen, three peak minima can be seen at 131 seconds, 209 seconds and 290 seconds.

FIG. 8 is a graph showing the measurement of the resistance (μΩ) of acetone as a function of time (seconds) in accordance with an aspect of the disclosure. As can be seen, three peak minima can be seen at 131 seconds, 209 seconds and 290 seconds.

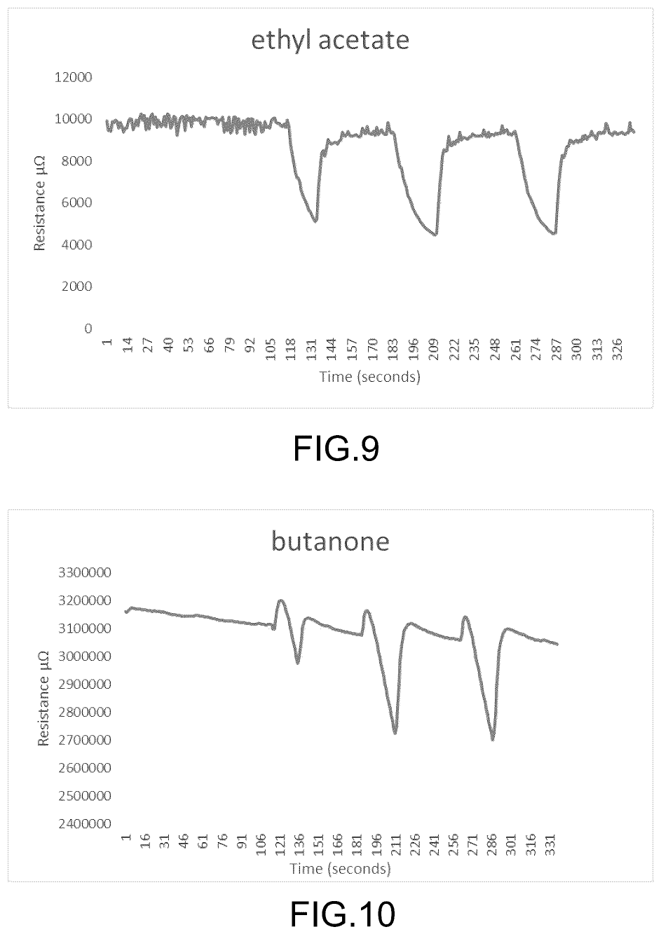

FIG. 9 is a graph showing the measurement of the resistance (μΩ) of ethyl acetate as a function of time (seconds) in accordance with an aspect of the disclosure. As can be seen, three peak minima can be seen at 131 seconds, 209 seconds and 290 seconds.

FIG. 10 is a graph showing the measurement of the resistance (μΩ) of butanone as a function of time (seconds) in accordance with an aspect of the disclosure. As can be seen, three peak minima can be seen at 131 seconds, 209 seconds and 290 seconds.

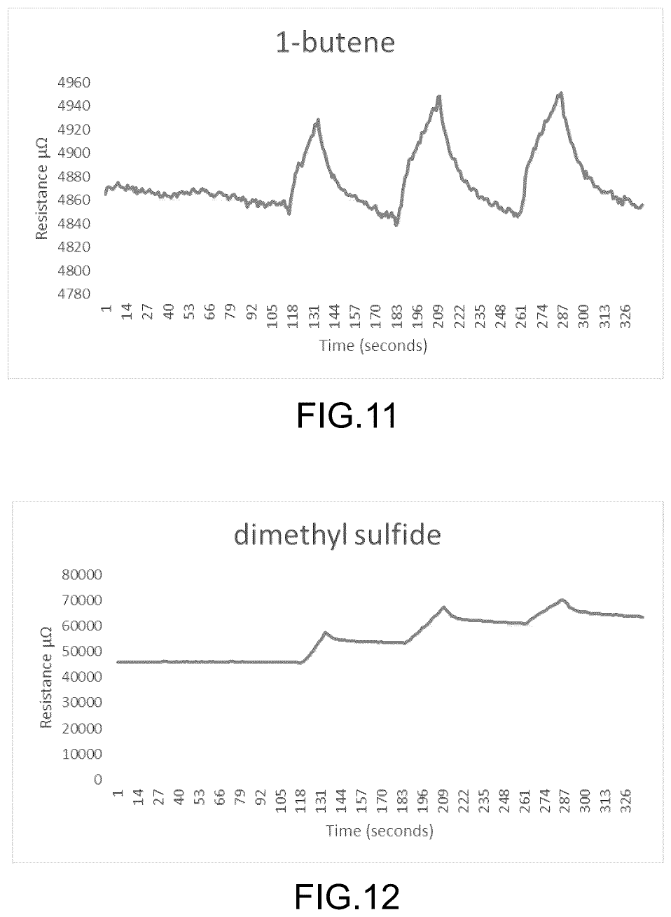

FIG. 11 is a graph showing the measurement of the resistance (μΩ) of 1-butene as a function of time (seconds) in accordance with an aspect of the disclosure. As can be seen, three peak maxima can be seen at 131 seconds, 209 seconds and 290 seconds.

FIG. 12 is a graph showing the measurement of the resistance (μΩ) of dimethyl sulfoxide as a function of time (seconds) in accordance with an aspect of the disclosure. As can be seen, three peak maxima can be seen at 131 seconds, 209 seconds and 290 seconds.

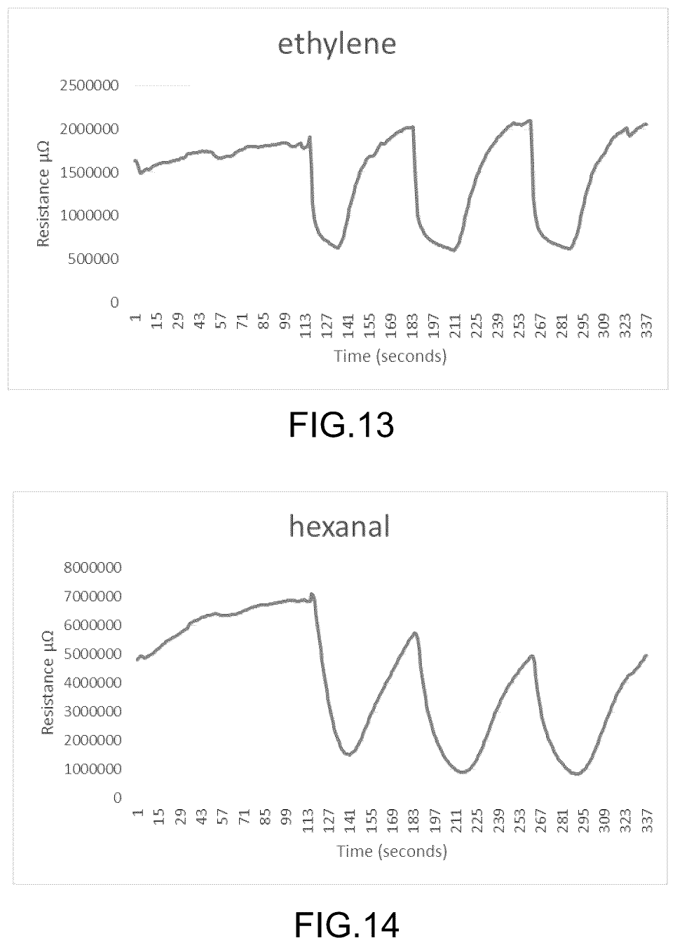

FIG. 13 is a graph showing the measurement of the resistance (μΩ) of ethylene as a function of time (seconds) in accordance with an aspect of the disclosure. As can be seen, three peak minima can be seen at 131 seconds, 209 seconds and 290 seconds.

FIG. 14 is a graph showing the measurement of the resistance (μΩ) of hexanal as a function of time (seconds) in accordance with an aspect of the disclosure. As can be seen, three peak minima can be seen at 131 seconds, 209 seconds and 290 seconds.

FIGS. 1 to 14 demonstrate that a wide range of VOCs, including those associated with the breath of a subject with influenza, as characterized by having peaks expressed at the same time intervals when the individual VOCs are analyzed. This demonstrates that influenza can be detected not by analyzing the magnitude of the individual VOC, but by measuring the magnitude of all the VOCs in the breath sample. This was demonstrated by performing measurements of breath samples with two different sensors. The results are tabulated in Tables 2 and 3 below.

TABLE 2
Results for Sensor 1.

| | Logistic Regression | | | Linear SVM | | |
|---|---|---|---|---|---|---|
| | Overall | | | Overall | | |
| Sensor 1 | accuracy | Sensitivity | Specificity | accuracy | Sensitivity | Specificity |
| A | 0.57 | 0.56 | 0.58 | 0.65 | 0.56 | 0.75 |
| B | 0.49 | 0.56 | 0.42 | 0.53 | 0.56 | 0.5 |
| C | 0.59 | 0.68 | 0.5 | 0.69 | 0.72 | 0.67 |
| D | 0.55 | 0.6 | 0.5 | 0.61 | 0.68 | 0.54 |
| E | 0.76 | 0.78 | 0.74 | 0.73 | 0.72 | 0.74 |
| F | 0.82 | 0.81 | 0.82 | 0.76 | 0.81 | 0.73 |
| | Nonlinear SVM | | | PLS-DA | | |
| | Overall | | | Overall | | |
| Sensor 1 | accuracy | Sensitivity | Specificity | accuracy | Sensitivity | Specificity |
| A | 0.47 | 0.48 | 0.46 | | | |
| B | 0.53 | 0.64 | 0.42 | 0.47 | 0.64 | 0.29 |
| C | 0.53 | 0.68 | 0.38 | 0.51 | 0.72 | 0.29 |
| D | 0.53 | 0.6 | 0.46 | 0.57 | 0.72 | 0.42 |
| E | 0.76 | 0.39 | 0.97 | 0.59 | 0.11 | 0.87 |
| F | 0.63 | 0 | 0.94 | 0.59 | 0.37 | 0.7 |

A: 3 peaks - 24 negative vs 25 positive
B: 1st peak - 24 negative vs 25 positive
C: 2nd peak - 24 negative vs 25 positive
D: 3rd peak - 24 negative vs 25 positive
E: 2^{nd} peak ct > 28 neg - 31 negative vs 18 positive
F: 2^{nd} peak ct > 25 neg - 33 negative vs 16 positive.

TABLE 3
Results for Sensor 2.

| | Logistic Regression | | | Linear SVM | | |
|---|---|---|---|---|---|---|
| | Overall | | | Overall | | |
| Sensor 2 | accuracy | Sensitivity | Specificity | accuracy | Sensitivity | Specificity |
| A | 0.6 | 0.75 | 0.43 | 0.47 | 0.58 | 0.33 |
| B | 0.6 | 0.75 | 0.43 | 0.6 | 0.75 | 0.43 |
| C | 0.6 | 0.75 | 0.43 | 0.58 | 0.71 | 0.43 |
| D | 0.64 | 0.79 | 0.48 | 0.53 | 0.62 | 0.43 |
| E | 0.64 | 0.67 | 0.63 | 0.69 | 0.67 | 0.7 |
| F | 0.78 | 0.69 | 0.83 | 0.78 | 0.75 | 0.79 |
| | Nonlinear SVM | | | PLS-DA | | |
| | Overall | | | Overall | | |
| Sensor 2 | accuracy | Sensitivity | Specificity | accuracy | Sensitivity | Specificity |
| A | 0.62 | 0.88 | 0.33 | | | |
| B | 0.62 | 0.96 | 0.34 | 0.55 | 1 | 0.05 |
| C | 0.58 | 0.92 | 0.19 | 0.55 | 1 | 0.05 |
| D | 0.58 | 0.92 | 0.19 | 0.55 | 1 | 0.05 |
| E | 0.6 | 0 | 0 | 0.53 | 0 | 0.89 |
| F | 0.64 | 0 | 0 | 0.6 | 0 | 1 |

A: 3 peaks - 21 negative vs 24 positive
B: 1st peak - 21 negative vs 24 positive
C: 2nd peak - 21 negative vs 245 positive
D: 3rd peak - 21 negative vs 245 positive
E: 1^{st} peak ct > 28 neg - 271 negative vs 18 positive
F: 1st peak ct > 25 neg - 29 negative vs 16 positive.

In the tables, linear SVM is where one can easily separate data with hyperplane by drawing straight line. In non-linear SVM the data cannot be easily separated by a straight line, and Kernal functions are used to make non-separable data into separable data. PLS-DA is partial least squares discrimination analysis, i.e., a supervised dimensionality reduction method that incorporates class labels in the analysis. It discovers latent variables (components) that maximize the separation between different predefined groups (e.g., healthy vs diseased) in the data.

The results indicate a positive correlation for the 14 VOCs measured. For example, the ambient air measurement of VOC at 1.75 parts per billion. Exhaled breath sample measurement of total VOC at 4.0 parts per billion. The calculation was as follows:

$$4. / 1.75 = 2.29$$

A ratio of 2.29 (+0.03) would indicate a positive result for a change in VOC, thus indicating the presence of influenza in the subject. A ratio of 2.3 will also indicate a positive result.

The results in Tables 2 and 3 show that the triple peaks correlate with influenza. The linear regression yields 82% accuracy, with a range of 79% to 85%. This can be compared to 50-70% for nasal swabs.

The present disclosure can include the following aspects:
I. A method for preparing, analyzing and characterizing volatile organic compounds (VOCs) from exhaled breath of humans that is useful for identifying the presence of an influenza infection, comprising:
   (a) extracting exhaled breath samples from humans to obtain the presence of specific VOCs or VOC(s) concentration levels.
   (b) producing isolated or measurable VOC concentrations extracted by:
      (i) VOC reaction reagents to identify or measure VOC concentration level.
      (ii) Selectively measuring attributes associated with VOCs:
         (a) measuring voltage differences using voltage measuring sensors or
         (b) selectively using size-based filtration to identify VOCs of interest.
         (c) analyzing exhaled breath samples produced.
II. The method of i, where the exhaled breath extracted is from spontaneous and awake humans.
III. The method of I, where the exhaled breath extracted is supported with man-made, mechanical devices used to assist with inhalation or exhalation.
IV. The method of i, where the analyzing VOCs comprises collecting human breath samples.
V. The method of i, where collecting comprises use of a mouthpiece, storage bag or intake port designed for exhaled breath capture.
VI. The method of i, where the method utilizes wirelessly connected, mobile, and reusable hardware to collect the breath samples from the user, patient or consumer.
VII. The method of i, where directly measuring the concentration and volume of the following VOCs from human exhaled breath:
Acetaldehyde,
propanal,
n-propyl acetate,
methyl-methacrylate,
styrene,
1,1-dipropoxypropane,
Isoprene,
acetone,
ethyl acetate,
butanone,
1-butene,
dimethyl sulfide,
ethylene,
hexanal.
VIII. The method of I, wherein directly measuring the concentration and volume of the following VOCs from external ambient air:
acetaldehyde,
propanal,
n-propyl acetate,
methyl-methacrylate,
styrene,
1,1-dipropoxypropane,
Isoprene,
acetone,
ethyl acetate,
butanone,
1-butene,
dimethyl sulfide,
ethylene,
hexanal.
IX. The method of I, wherein the humidity of the sample remains constant with a minimum of 50% and a maximum of 80%. Along with maintaining standard temperature and pressure (Temperature: 25 degrees Celsius (298.15 degrees Kelvin) Pressure: 1 atm (101.325 kPa) of both the sample gas and ambient air.
X. The method of I, where analyzing comprises using ratio analysis of the following fourteen VOCs:
acetaldehyde,
propanal,
n-propyl acetate,
methyl-methacrylate,
styrene,
1,1-dipropoxypropane,
Isoprene,
Acetone,
ethyl acetate,
butanone,
1-butene,
dimethyl sulfide,
ethylene,
hexanal.
where the measured concentrations from the sample exhaled breath and the external ambient air, in which there is between a 1.1× and 5× ratio fluctuation between the sample VOC measurements and the ambient air measurements. By taking measurements of the ambient air as well as the exhaled breath sample gas and a positive ratio is calculated for at least seven of the fourteen specified VOC listed, a positive test can be determined for the presence of influenza virus.
XI. The method of I, wherein if the results of comparing the listed VOCs concentrations between ambient air and exhaled breath in seven of the fourteen VOC biomarkers, a positive test can be resulted which identifies an influenza infection.

### REFERENCES

Traxler, S., Bischoff, AC., Saß, R. et al. VOC breath profile in spontaneously breathing awake swine during influenza A infection. Sci Rep 8, 14857 (2018). https://doi.org/10.1038/s41598-018-33061-2
Traxler, Selina, et al. “Volatile scents of influenza A and S. pyogenes (co-) infected cells.” Scientific reports vol. 9, 118894. 11 Dec. 2019, doi: 10.1038/s41598-019-55334-0
Traxler S, Bischoff A C, Trefz P, Schubert J K, Miekisch W. Versatile set-up for non-invasive in vitro analysis of headspace VOCs. J Breath Res. 2018 Jul. 3; 12 (4): 041001. doi: 10.1088/1752-7163/aaccad. PMID: 29900878
Aksenov, Alexander A et al. “Cellular scent of influenza virus infection.” Chembiochem: a European journal of chemical biology vol. 15, 7 (2014): 1040-8. doi: 10.1002/cbic.201300695
Mark Woollam, Paula Angarita-Rivera, Amanda P Siegel, Vikas Kalra, Rajat Kapoor, Mangilal Agarwal, Exhaled VOCs can discriminate subjects with COVID-19 from healthy controls, Journal of Breath Research, 10.1088/1752-7163/ac696a, 16, 3, (036002), (2022).
Smolinska A, Klaassen E M, Dallinga J W, van de Kant K D, Jobsis Q, Moonen E J, van Schayck O C, Dompeling E, van Schooten F J. Profiling of volatile organic compounds in exhaled breath as a strategy to find early predictive signatures of asthma in children. PLOS One. 2014 Apr. 21; 9 (4): e95668. doi: 10.1371/journal.pone.0095668. Erratum in: PLOS One. 2014; 9 (8): e105447. PMID: 24752575; PMCID: PMC3994075.
Peter J. Mazzone, Analysis of Volatile Organic Compounds in the Exhaled Breath for the Diagnosis of Lung Cancer, Journal of Thoracic Oncology, Volume 3, Issue 7, 2008, Pages 774-780, ISSN 1556 0864, https://doi.org/10.1097/JTO.0b013e31817c7439.
(https://www.sciencedirect.com/science/article/pii/S1556086415333529)
Dospinescu, Tiele. “Sniffing Out Urinary Tract Infection—Diagnosis Based on Volatile Organic Compounds and Smell Profile.” Biosensors (Basel) 10.8 (2020): 83-. Web.
Schnabel, R., Fijten, R., Smolinska, A. et al. Analysis of volatile organic compounds in exhaled breath to diagnose ventilator-associated pneumonia. Sci Rep 5, 17179 (2015). https://doi.org/10.1038/srep17179
Miekisch, W., Schubert, J. K. & Noeldge-Schomburg, G. F. Diagnostic potential of breath analysis-focus on volatile organic compounds. Clin Chim Acta. 347, 25-39 (2004).
Boots, A. W. et al. The versatile use of exhaled volatile organic compounds in human health and disease. J. Breath Res. 6, 027108 (2012).
Libardoni M, Stevens P T, Waite J H and Sacks R 2006 Analysis of human breath samples with a multi-bed sorption trap and comprehensive two-dimensional gas chromatography (GCxGC) J. Chromatogr. B 842 13-21
Miekisch W, Schubert J K and Noeldge-Schomburg G F 2004 Diagnostic potential of breath analysis-focus on volatile organic compounds Clin. Chim. Acta 347 25-39
Fens N, Zwinderman A H, van der Schee M P, de Nijs S B, Dijkers E, Roldaan A C, Cheung D, Bel E H, and Sterk P J 2009 Exhaled breath profiling enables discrimination of chronic obstructive pulmonary disease and asthma Am. J. Respir. Crit. Care Med. 180 1076-82
Zethelius B, Berglund L, Sundstrom J, Ingelsson E, Basu S, Larsson A, Venge P and Arnlov J 2008 Use of multiple biomarkers to improve the prediction of death from cardiovascular causes N. Engl. J. Med. 358 2107-16
Benirschke R C, McElvania E, Thomson R B Jr, Kaul K L, Das S. Clinical Impact of Rapid Point-of-Care PCR Influenza Testing in an Urgent Care Setting: a Single-Center Study. J Clin Microbiol. 2019 Feb. 27; 57 (3): e01281-18. doi: 10.1128/JCM.01281-18. PMID: 30602445; PMCID: PMC6425177.
Tibor Pasinszki, Melinda Krebsz, Chapter One-Advances in celiac disease testing, Editor(s): Gregory S. Makowski, Advances in Clinical Chemistry, Elsevier, Volume 91, 2019, Pages 1-29, ISSN 0065-2423, ISBN 9780128174715, https://doi.org/10.1016/bs.acc.2019.03.001.
F. Di Francesco, R. Fuoco, M. G. Trivella, A. Ceccarini, Breath analysis: trends in techniques and clinical applications, Microchemical Journal, Volume 79, Issues 1-2, 2005, Pages 405-410, ISSN 0026-265X, https://doi.org/10.1016/j.microc.2004.10.008.
(https://www.sciencedirect.com/science/article/pii/S0026265X04001973)
Lu, Yijang, et al. “Fabrication of Carbon-Nanotube-Based Sensor Array and Interference Study.” Journal of Materials Research, vol. 26, no. 16, 23 Aug. 2011, pp. 2017-2023, https://doi.org/10.1557/jmr.2011.225. Accessed 11 Apr. 2022.
Li, Jing, et al. Nanotechnology Based Cell-All Phone-Sensors for Extended Network Chemical Sensing. 1 Oct. 2012, https://doi.org/10.1109/icsens.2012.6411055. Accessed 8 Jun. 2023.
Li, Jing, et al. “Carbon Nanotube Sensors for Gas and Organic Vapor Detection.” Nano Letters, vol. 3, no. 7, July 2003, pp. 929-933, https://doi.org/10.1021/nl034220x. Accessed 30 Jun. 2019.

While various aspects have been described above, it should be understood that they have been presented by way of example only, and not limitation. Thus, the breadth and scope of an aspect of the present invention should not be limited by any of the above-described exemplary aspects, but should be defined only in accordance with the following claims and their equivalents.

What is claimed is:

1. A method for preparing, analyzing and characterizing volatile organic compounds (VOCs) from exhaled breath of subjects that is useful for identifying a presence of an influenza infection, comprising:
(a) extracting exhaled breath samples from subjects to obtain a presence of specific VOCs or VOC(s) concentration levels; and
(b) producing isolated or measurable VOC concentrations extracted by:
(i) VOC reaction reagents to identify or measure VOC concentration level,
(ii) selectively measuring attributes associated with VOCs,
(a) measuring voltage differences using voltage measuring sensors or resistance sensor, or
(b) selectively using size-based filtration to identify VOCs of interest, and
(c) analyzing exhaled breath samples produced.

2. The method of claim 1, wherein a positive result for influenza is a ratio of about 2.29 sample measurement/ambient air measurement.

3. The method of claim 1, having an accuracy in a range of about 79% to about 85% for influenza.

4. The method of claim 1, wherein the VOCs are at least one selected from the group consisting of acetaldehyde, propanal, n-propyl acetate, methyl-methacrylate, styrene, 1,1-dipropoxypropane, isoprene, acetone, ethyl acetate, butanone, 1-butene, dimethyl sulfide, ethylene and hexanal.

5. The method of claim 1, wherein the VOCs are detected by resistance measurement.

6. The method of claim 1, wherein in VOCs are detected by resistance measurement, and the resistance measurement shows peaks at about 131 seconds, about 209 seconds and about 290 seconds.

7. The method of claim 1, wherein the breath samples are collected by exhaling into a polyvinyl fluoride gas sampling bag.

8. The method of claim 1, wherein the breath samples are selectively bound to carbon nanotubes.

9. The method of claim 1, further comprising:
selectively binding the breath samples to carbon nanotubes;
measuring a baseline resistance; and
comparing the baseline resistance to resistance changes after sample collection.

10. The method of claim 1, wherein the voltage differences are measures using metal-oxide semiconductors, conductive polymers or polymer composites containing carbon black.

11. A method for preparing, analyzing and characterizing volatile organic compounds (VOCs) from exhaled breath of humans for identifying a presence of an influenza infection, comprising:
(a) extracting exhaled breath samples from subjects to obtain a presence of total VOC concentration levels; and
(b) Selectively measuring attributes associated with the total VOCs by measuring voltage differences using voltage measuring sensors or resistance sensors.

12. The method of claim 11, wherein a positive result for influenza is a ratio of about 2.29 sample measurement/ambient air measurement.

13. The method of claim 11, having an accuracy in a range of about 79% to about 85% for influenza.

14. The method of claim 11, wherein the total VOCs include at least one VOC selected from the group consisting of acetaldehyde, propanal, n-propyl acetate, methyl-methacrylate, styrene, 1,1-dipropoxypropane, isoprene, acetone, ethyl acetate, butanone, 1-butene, dimethyl sulfide, ethylene and hexanal.

15. The method of claim 11, wherein the VOCs are detected by resistance measurement.

16. The method of claim 11, wherein in VOCs are detected by resistance measurement, and the resistance measurement shows peaks at about 131 seconds, about 209 seconds and about 290 seconds.

17. The method of claim 11, wherein the breath samples are collected by exhaling into a polyvinyl fluoride gas sampling bag.

18. The method of claim 11, wherein the breath samples are selectively bound to carbon nanotubes.

19. The method of claim 11, further comprising:
selectively binding the breath samples to carbon nanotubes;
measuring a baseline resistance; and
comparing the baseline resistance to resistance changes after sample collection.

20. The method of claim 1, wherein the voltage differences are measures using metal-oxide semiconductors, conductive polymers or polymer composites containing carbon black.
